(19) United States
(12) Patent Application Publication
(10) Pub. No.: US 2024/0033440 A1
(43) Pub. Date: Feb. 1, 2024

(54) END OF DOSE MECHANISM

(71) Applicant: Owen Mumford Limited, Woodstock, Oxfordshire (GB)

(72) Inventors: Toby Ken COWE, Woodstock, Oxfordshire (GB); Edward Thomas George BARRETT, Woodstock, Oxfordshire (GB)

(21) Appl. No.: 18/264,128

(22) PCT Filed: Feb. 3, 2022
(86) PCT No.: PCT/EP2022/052573

§ 371 Date: Aug. 3, 2023

(30) Foreign Application Priority Data
Feb. 4, 2021 (GB) 2101582.1

(51) Int. Cl. A61M 5/315

(52) U.S. Cl. CPC A61M 5/3157

(57) ABSTRACT

Aspects of the present invention relate to an injection device comprising a cartridge arranged to contain a medicament and piston and a drive mechanism arranged to move the piston from a first position where medicament is contained within the syringe and a second position in which the medicament has been delivered from the cartridge to a user. The drive mechanism includes a flexible portion which co-operates with another part of the injection device such that the flexible portion is moved towards the central axis of the injection device during a portion of the movement of the drive mechanism from the first position to the second position and disengages from the another part of the injection device at or near the second position to indicate that the medicament has been delivered from the cartridge.

### TECHNICAL FIELD

The present disclosure relates to an end of dose mechanism for an injection device.

### BACKGROUND

Injection devices are used for the convenient administration of medicaments to patients. For example, injection devices, which may be auto-injectors, may be used for providing a single metered dose of a medicament. Such devices may be either single use “disposable” devices in which the device is typically provided with a syringe already installed, and which is not user-replaceable, or “reusable” devices which allow the user to replace the syringe when the medicament has been used.

Injection devices generally comprise a firing mechanism that is arranged to cause a fluid to be delivered from the syringe automatically under the force of a drive mechanism, such as a drive spring. Optionally, injection devices may also comprise an insertion mechanism to displace the syringe within a housing of the injection device to cause needle penetration. The delivery arrangement generally acts via a plunger which includes a plunger and may also include or engage a piston (also referred to as a “bung”) which is slidably provided within the syringe.

It is often desirable to indicate to a user of the injection device when delivery of the medicament in the syringe has been completed. To achieve this an end of dose indicator is provided in the injection device.

### SUMMARY OF THE INVENTION

Aspects and embodiments of the invention provide an end of dose indicator for an injection device as claimed in the appended claims.

According to an aspect of the present invention there is provided an injection device including: a cartridge arranged to contain a medicament and piston; a drive mechanism arranged to move the piston from a first position where medicament is contained within the syringe and a second position in which the medicament has been delivered from the cartridge to a user; wherein the drive mechanism includes a flexible portion which co-operates with another part of the injection device such that the flexible portion is moved towards the central axis of the injection device during a portion of the movement of the drive mechanism from the first position to the second position and disengages from the another part of the injection device at or near the second position to indicate that the medicament has been delivered from the cartridge. Incorporating the end of dose mechanism into the drive mechanism removes the need for a separate end of dose mechanism.

Preferably the drive member comprises a plunger configured to contact the piston at a distal end and a drive spring configured to move the plunger to cause the piston to move from the first position to the second position.

Optionally, the plunger includes the flexible portion and the flexible portion comprises a flexible finger which is connected to the plunger at a first end and extends outwardly away from the central axis of the injection device.

Optionally, the plunger comprises a distal elongate portion and a proximal head portion wherein the flexible finger is connected to the plunger at the distal end of the head portion.

The flexible finger may be a lateral projection. The flexible portion may extend rearwardly from the distal end of the plunger alternatively, the flexible portion may be an extension from the distal end of the spring. The flexible portion may include a lateral projection. The lateral projection may be hook shaped.

Optionally, the flexible portion extends rearwardly from the distal end of the spring.

Preferably the injection device further includes a housing and the another part of the injection device is a portion of housing. The portion of the housing may include an inclined surface of an inner wall of the housing extending along the path of travel of the flexible portion from the first position to the second position.

According to another aspect of the present invention there is provided a plunger for use in an injection device.

According to a further aspect of the present invention there is provided a drive spring for use in an injection device.

### BRIEF DESCRIPTION OF THE DRAWINGS

One or more embodiments of the invention will now be described, by way of example only, with reference to the accompanying drawings, in which:

FIG. 1 illustrates an auto-injector according to a first embodiment of the present invention;

FIG. 2 illustrates an end of dose mechanism according to a first embodiment of the present invention;

FIG. 3 illustrates an auto-injector according to a first embodiment of the present invention where medicament has been delivered to the user; and

FIG. 4 illustrates an end of dose mechanism according to a second embodiment of the present invention.

### DETAILED DESCRIPTION

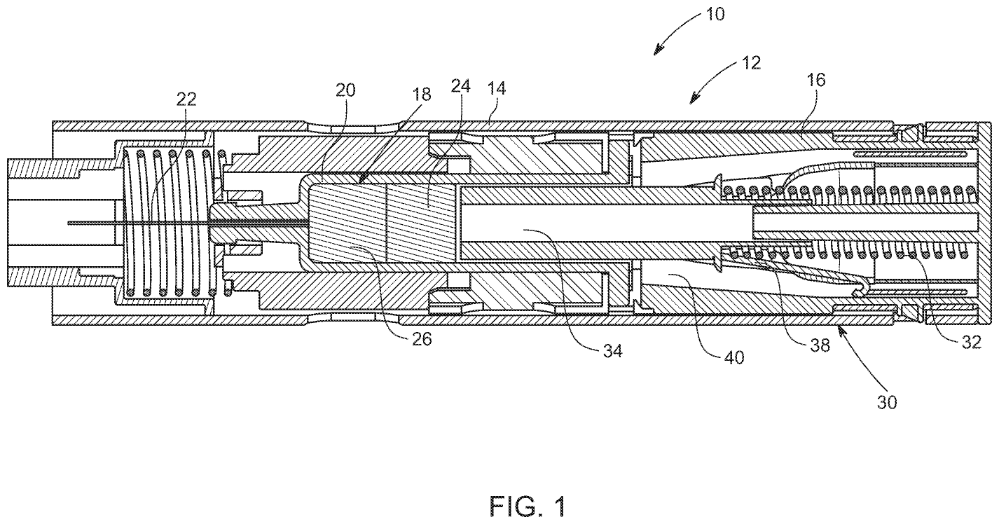

An autoinjector incorporating the end of dose mechanism according to an embodiment of the present invention is described herein with reference to FIG. 1.

With reference to FIG. 1, the auto-injector 10 comprises a housing 12 including a distal housing portion 14 and a proximal housing portion 16. The housing 12 is shaped to receive a syringe 18 comprising a body 20, a needle 22 at its distal end and a piston 24 at its proximal end. The syringe 18 is configured to contain medicament 26 between the needle 22 and the piston 24.

The auto-injector 10 is further provided with a drive mechanism 30 configured to cause the medicament 26 contained in the syringe 18 to be delivered to a user when the auto-injector 10 is activated. The auto-injector may be activated by any suitable means, for example a trigger button on the side of the housing, a trigger button on the proximal end of the housing or contact activation caused by a needle shroud being pushed into the auto-injector by contact with the injection site.

The drive mechanism 30 comprises a drive spring 32 and a plunger 34. Activation of the drive mechanism 30 causes the spring, in this instance a compression spring, to be released. Energy stored within the drive spring 32 causes the distal end of the drive spring 32 to move distally away from the proximal end of the auto-injector 10.

The distal end of the drive spring 32 places a force on the plunger 34 of the auto-injector thereby moving the plunger 34 distally within the auto-injector. The plunger 34 is in contact with the piston 24 of the syringe 18. Thus, force is placed on the piston 24 moving the piston 24 distally within the syringe 18 causing medicament 26 to be delivered to the user through the needle 22.

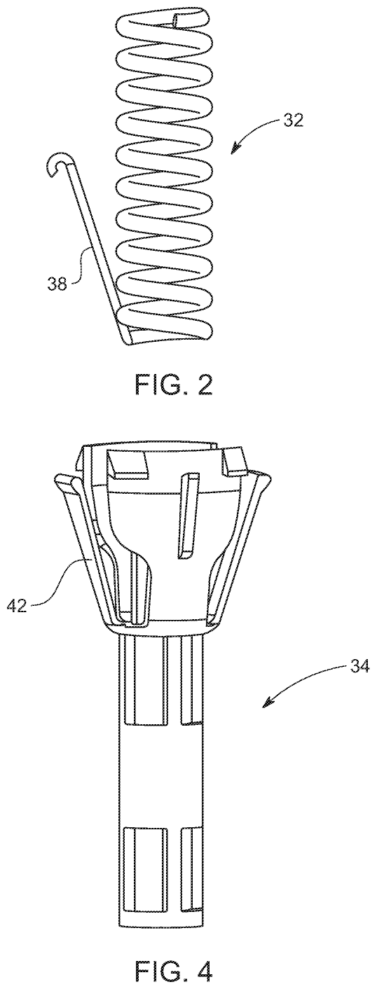

FIG. 2 illustrates an end of dose mechanism according to a first embodiment of the present invention. In the first embodiment the drive spring 32 is provided with an extension 38 at its distal end. The extension 38 comprises a length of the spring material which extends laterally and proximally from the central axis of the spring. As the distal end of the spring moves distally during use of the auto-injector 10 the extension also moves distally.

Referring back to FIG. 1, it can be seen that the proximal housing portion 16 is provided with an angled inner surface 40. The inner surface 40 is angled so that the proximal part of the surface is a greater distance from the central axis of the auto-injector to the distal part of the angled surface 40. This means that, as the extension 38 moves distally during delivery of the medicament, the extension 38 is flexed so that the distance of the end portion of the extension moves closer to the central axis of the auto-injector.

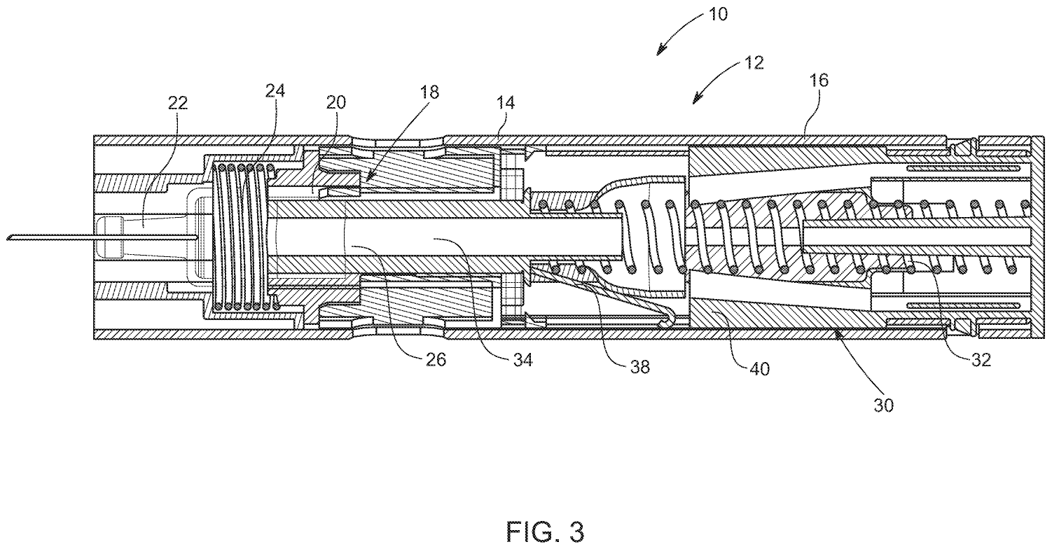

When the spring is at its fullest extension at/near full delivery of the medicament the end portion of the extension passes past the end of the angled inner surface 40. This is shown in FIG. 3. Once the extension has passed the end of the angled inner surface 40 the energy stored within the extension causes the end of the extension to move away from the central axis of the auto-injector and hit the inner wall of the housing. Hitting the inner wall of the housing causes the end of the extension to make a noise indicating the end of delivery of the medicament.

Although the present invention has been described with reference to an extension which is integral to the spring itself the skilled person will understand that the extension may be provided by a separate member which is attached to the distal end of the spring and extends rearwardly towards the proximal part of the auto-injector.

FIG. 4 illustrates a second embodiment of the present invention. In this embodiment like reference numerals will denote like features. In the second embodiment the plunger 34 is provided with a rearwardly extending arm 42 which, in use, extends from a distal end where it is connected to the plunger to a free proximal end.

As described previously with reference to embodiment 1, the proximal housing portion 16 is provided with an angled inner surface 40. The inner surface 40 is angled so that the proximal part of the surface is a greater distance from the central axis of the auto-injector to the distal part of the angled surface 40. As the plunger 34 moves distally under the force of the drive spring 32 the arm 42 moves along the angled inner surface 40 and thus towards the central axis of the auto-injector.

At or near the end of delivery of the medication the end of the arm will pass over the end of the angled inner surface 40. When the end of the arm passes over the end of the angled inner surface the energy stored within the arm causes the end of the arm to move away from the central axis of the auto-injector and hit the inner wall of the housing. Hitting the inner wall of the housing causes the end of the arm to make a noise indicating the end of delivery of the medicament.

The free end of the extension of the plunger arm or spring arm may be provided with an alternative material to increase the sound made on hitting the housing. And/or the housing may be provided with a different insert/made of a different material.

Additionally, the free end of the extension of the plunger arm or spring arm may be provided with a lateral extension extending away from the central axis of the plunger arm or spring arm. This lateral extension may take any suitable form, for example it may be a right angled extension as illustrated in FIG. 4 or be a hook as illustrated in FIG. 2. The free extension may be dimensioned and/or shaped to increase the amount of flexion of the arm towards the central axis of the auto-injector and/or to increase the sound made on contact with the housing.

Although the present invention has been described with reference to the proximal housing portion being provided with an inclined surface the skilled person will understand that the inclined surface may be provided by any internal surface within the auto-injector.

For example, the device may be provided with an insert internal to the housing which provides the inclined surface or the inclined surface may be provided on the distal portion of the auto-injector.

Although the present invention has been described with reference to the auto-injector illustrated and described with reference to FIG. 1. It will be understood by the skilled person that it may be implemented in any relevant injection device where a plunger or drive spring is used including, but not limited to, an injection device comprising a single housing, a reusable auto-injector, and an injection device having an upper and a lower housing part.

Additionally, although the auto-injector has been described as receiving a syringe with a body, needle and a piston the invention may be implemented in a device configured to receive a cartridge having a septum at its distal end which is suitable, in use, to be pierced by a needle to allow the medicament contained within the cartridge to be delivered to a user.

Certain terminology is used in the following description for convenience only and is not limiting. The word “distal” refers to the front, or patient, end of the device. The word “proximal” refers to the rear, or syringe user, end of the device. The term “longitudinal”, with or without axis, refers to a direction on an axis through the device in the direction of the longest extension of the device. The term “radial” or “transverse/transversal”, with or without axis, refers to a direction generally perpendicular to the longitudinal direction, e.g. “radially outward” would refer to a direction pointing away from the longitudinal axis.

It should be understood that the detailed description and specific examples are given by way of illustration only, since various changes and modifications within the spirit and scope of the disclosure will become apparent to those skilled in the art from the description.

1. An injection device comprising:
i. a housing comprising an inner wall having an inclined surface extending along the path of travel;
ii. a cartridge arranged to contain a medicament and piston
iii. a drive mechanism arranged to move the piston from a first position where medicament is contained within the cartridge and a second position in which the medicament has been delivered from the cartridge to a user;
wherein the drive mechanism includes a flexible portion which co-operates with another part of the injection device such that the flexible portion is moved towards a central axis of the injection device during a portion of the movement of the drive mechanism from the first position to the second position and disengages from the inclined surface at or near the second position to contact a surface of the housing thereby audibly indicating that the medicament has been delivered from the cartridge.

2. An injection device according to claim 1 wherein the drive member comprises a plunger configured to contact the piston at a distal end and a drive spring configured to move the plunger to cause the piston to move from the first position to the second position.

3. An injection device according to claim 2 wherein the plunger includes the flexible portion and the flexible portion comprises a flexible finger which is connected to the plunger at a first end and extends outwardly away from the central axis of the injection device.

4. An injection device according to claim 3 wherein the plunger comprises a distal elongate portion and a proximal head portion wherein the flexible finger is connected to the plunger at a distal end of the head portion.

5. An injection device according to claim 3 wherein the flexible finger includes a lateral projection.

6. An injection device as claimed in claim 3 wherein the flexible portion extends rearwardly from the distal end of the plunger.

7. An injection device as claimed in claim 1 wherein drive mechanism comprises a spring and the flexible portion comprises an extension from the distal end of the spring.

8. An injection device as claimed in claim 7 wherein the flexible portion includes a lateral projection.

9. An injection device as claimed in claim 8 wherein the lateral projection is hook shaped.

10. An injection device as claimed in claim 7 wherein the flexible portion extends rearwardly from the distal end of the spring.

11-14. (canceled)
